(19) United States
(12) Patent Application Publication
(10) Pub. No.: US 2004/0030365 A1
(43) Pub. Date: Feb. 12, 2004

(54) Medical device to restore functions of a fibrillating heart by cardiac therapies remotely directed by a physician via two-way communication

(76) Inventor: Leo Rubin, Suffern, NY (US)

Correspondence Address: LEHNHARDT & ASSOCIATES, 20 NASSAU ST., SUITE 225, PRINCETO, NY, 08542, US

(21) Appl. No.: 10/203,544

(22) PCT Filed: Nov. 30, 2001
(86) PCT No.: PCT/US01/44578

(51) Int. Cl. A61N 1/08

(52) U.S. Cl. 607/60

(57) ABSTRACT

The present invention relates generally to a medical device for monitoring and controlling arrhythmias, and for restoring functions of a fibrillating heart in a subject by therapies directed by a physician via two-way communication between the device and the physician, comprising (a) an implant; and (b) a wearable unit comprising a programmer, and a position-locating module; (c) a first interface for communication between the implant and the programmer, and (d) a second interface for two-way communication between a physician in an emergency team and the programmer through the position-locating module, wherein data on the location of the subject and the subject's heart conditions are sent to the physician for evaluation and instructions for therapy are sent from the physician to the programmer. The present invention also provides for a method of emergency treatment of a subject

### FIELD OF THE INVENTION

The present invention relates generally to medical devices for monitoring and for emergency treatment of a cardiac arrest patient or subject to restore effective cardiac function. The medical device can be used for monitoring and controlling arrhythmia, and for restoring functions of a fibrillating heart in a subject by therapies directed by a physician via two-way communication between the device and the physician, and it contains (a) an implant; (b) a wearable unit comprising a programmer, and a position-locating module; (c) a first interface for communication between the implant, and the programmer; and (d) a second interface for two-way communication between a physician in an emergency team and the programmer through the position-locating module.

### BACKGROUND OF THE INVENTION

Heart disease is the leading cause of death in the industrially developed world. Nearly two thirds of patients die from coronary disease before they reach a hospital. In the United States, sudden death claims about 1,200 lives daily. Almost 25% of sudden death victims are usually healthy adults without any symptoms of heart disease before this abrupt terminal event. Survival rates from out of hospital cardiac arrests are dismal, with published reports ranging from 0-18%. Eisenberg et al. (1990) Ann. Emerg. Med. 19:179-186.

Cardiac arrest is not a simple binary event but a complex electromechanical, neurohumoral, sudden catastrophic occurrence that can frequently be aborted or reverted by applying an electrical shock. However, frequently events leading up to and immediately following cardiac arrest require a much more elaborate intervention in order to accomplish successful resuscitation.

Cardiac arrest occurs when there is electrical or mechanical dysfunction in the heart, and results in the heart failing to pump blood, causing a lack of oxygen to the brain. The contributing factors that can result in cardiac arrest are many, and include hypertension , diabetes, obesity, aging, and drug use. Cardiac arrest results in administration of proper medications. Mortality is higher if treatment is delayed, but the prognosis is significantly improved if vigorous treatment begins immediately. Thus far drug therapies have had little effect on the mortality rate due to cardiac arrest. The primary goal during the past three decades has been to teach cardiopulmonary resuscitation (CPR) techniques to as many people as possible in an attempt to increase the percent of survivors.

The anatomy of the heart is described and illustrated in detail in numerous reference works on anatomy and cardiac surgery, including standard texts such as Surgery of the Chest (Sabiston and Spencer, eds., Saunders Publ., Philadelphia). Basically, the heart has a special system of muscles that cause the heart tissue to regularly and continuously contract. The heart has two major pumping chambers in the heart, the left and right ventricles. Simultaneously contracting, these chambers expel blood into the aorta and the pulmonary artery. Blood enters the ventricles from the left and right atria, respectively. The atria are small antechambers which contract in a separate action which precedes the major ventricular contraction by an interval of about 100 milliseconds (ms), known as the AV delay. The contractions arise from electrical excitation or depolarization waves which begin in the right atrium and spread to the left atrium. The excitation then enters the atrio-ventricular node which delays its passage via the Bundle of His into the ventricles. The heart tissue contracts following its depolarization. The Bundle of His regulates the speed of depolarization from the atria to the ventricles. All muscle tissue surrounding a specific compartment simultaneously contracts and the atria and ventricles contract in the proper time sequence. One complete contraction of both the atria and the ventricles constitutes a beat.

However, for patients with heart problems, the depolarization carried through the heart tissue may become irregular or chaotic (fibrillation), causing the heart to beat unevenly or to stop beating which may cause severe injuries or death. The detection and monitoring of the heart problem is based on the appearing in the electrocardiogram a small signal known as the P-wave that precedes the atrial contraction while a much larger signal, known as the QRS complex, with a respective heart chambers. Further, the atrial and ventricular waves (“A-wave and R-wave” respectively) can be detected using internal electrodes which are in contact with the heart muscle. They are different in amplitude, shape and in the time of occurrence, from the P-wave and R-wave of the ECG. The A-wave and V-wave are dependent upon the specification location of the electrodes, their contact with muscle, size, shape, reference electrode, etc.

As used herein the term “implant” encompasses implantable defibrillators, implantable pacer, implantable injector, etc. Implant also includes an implantable stimulator which can be used to treat/prevent epileptic seizure The revival of normal heart beat can be accomplished by a process called defibrillation which was developed and used over the past four decades. To defibrillate a heart, an intense pulse of electrical current, a defibrillation pulse is applied to the heart. This electrical defibrillation pulse depolarizes the muscle fibers of the heart and, thus enable the heart to return to normal beating. Moreover, implantable defibrillators and arrhythmia controlling pacemakers, to restore adequate heart function in a chaotically pumping heart, have also been developed and entered into wide clinical use. Further, thyroid hormones and their analogs have also been used to treat heart failure, as described in U.S. Pat. No. 5,158,978.

The typical implantable defibrillator incorporates a pacemaker that supplies pulses when the heart does not contract on its own, it restores missing beats. The pulses are typically delivered via a pacing lead attached to the ventricle. The ventricular depolarization can be sensed by the same lead. An additional lead contacts the atrium to sense atrial depolarization, or “P-waves,” if desired. In AV sequential pacers, discussed below, the atrial lead is also used for atrial stimulation.

Implantable defibrillators, combined with pacemakers, are useful in treating a number of cardiac disorders such as heart block caused by impairment of the ability of the Bundle of His to conduct normal excitation from the atrium to the ventricle. The implantable defibrillator itself is a battery powered, hermetically sealed, completely self-contained electronic device which is implanted in the body at a suitable site such as the pectoral or axillary region within an inch from the surface of the defibrillator. The proximal end of the lead is taken out through an opening in the blood vessel and electrically connected to the defibrillator. There are usually four or more electrodes involved in an implantable defibrillator-cardioverter-pacer with its leads reaching the heart via the venous route. These electrodes include (1) one (unipolar) or two (bipolar) electrodes in contact with the right ventricle to sense and stimulate. If a single electrode is used, then the electrical circuit is completed through a large area “indifferent” or “ground” electrode, usually the metal housing of the implant; (2) one (unipolar) or two (bipolar) electrodes in contact with the right atrium to sense and stimulate, as described in (1); and (3) one large electrode, not necessarily in contact with the right ventricle but within the heart, for delivery of the defibrillation pulse. The return electrode for the current is provided either by an additional large surface electrode within the heart, possibly in the right atrium, or the metal housing of the implant. Earlier, the preferred placement for the defibrillation electrode(s) was on the outside of the heart. For instance, a truncated cone-shaped cup was sewed to the pericardial sac with its counterpart within the right heart. Alternatively, two “patches” were sewn onto the ventricle with the defibrillation pulse passed through the heart between those electrodes. Inside the defibrillator, the stimulation pulses are formed by a pulse generator.

In the past, pulse generators have taken several forms but fall into two general categories: (1) those where the pulse generator consists of an R-C timing circuit and (2) those where oscillations in the output of a high frequency clock (R-C or crystal oscillator) are counted by digital circuitry. In circuits of the second kind, the pulse generator typically comprises a digital counter and logic circuitry for producing an output pulse when a given number of clock pulses is counted and means for resetting the counter in response to spontaneous or stimulated activity. An early example is found in U.S. Pat. No. 3,557,796 to Keller et al. With the miniaturization of stored program data processors, microprocessor cardiac defibrillation systems have given rise to more complex and yet more flexible counting arrangements.

For example, a cardiac period number may be placed into a register which is of a stimulation pulse, as in “Multi-Mode Microprocessor-Based Programmable Cardiac Pacer” U.S. patent application Ser. No. 207,003, filed Nov. 14, 1980 by Leckrone et al, and incorporated herein by reference in its entirety.

The level of electrical stimulation is very important since the charge density in the myocardium surrounding the bare electrode at the distal end of the lead determines whether the cardiac muscle depolarizes and contracts, or not. The electrical pulses for defibrillation and demand pacing are typically delivered to the heart through different methods. The defibrillation pulse from an implanted device has historically been delivered through a large area electrical patch sewn to the exterior surface of the heart. Another electrode may be placed inside the heart or elsewhere within the body, or the heart or under the skin. The electrical patch and its counter-part are connected to a capacitor that is charged by a battery, through a connector to produce high voltage, to be capable of delivering an electrical defibrillation pulse between the contacts with tissues. Once the capacitor discharges the defibrillation pulse, the current enters directly into the heart of the subject so as to defibrillate the heart. The pulse then exits the tissues through the counter electrode.

Several factors are known to affect stimulation, including the wave form of the stimulation pulse, the voltage, the duration of the stimulation or “pulse width,” the type of electrode including the area of contact and the resistance of the contacting tissue and electrochemical factors as well as the type of lead system used, i.e. unipolar or bipolar. In unipolar systems, the return terminal is on the conductive housing of the implant. In bipolar systems, the end of the lead contains two spaced contacts, one of which would be regarded as the reference or return electrode and the other is called the reference electrode for sensing and the return electrode for stimulation.

Advances in defibrillator development have enabled pulse parameters such as rate, pulse width and amplitude to be altered by an externally generated programming signal, for example, using a succession of magnetic pulses to actuate a tiny reed switch within the defibrillator. Once a defibrillator is implanted and in parameter information, to ascertain the exact levels of those parameters without a programmer (a two-way short range telecontroller) that is capable of interrogating the implant and interpreting its response.

The use of pacer catheter makes it possible to apply demand pacing pulses to the heart on demand as needed to maintain an acceptable heart rate. The pacer catheter is a long flexible probe, usually made of silastic or polyurethane, with electrical leads running the length of the catheter within. At one end of the probe, the leads are connected to an exposed metal surface, the electrode. In a bipolar system, part way up the probe, the lead is connected to a second exposed metal surface called a return electrode. Finally, at the other end of the probe, the lead is connected to a regulator that has a controller for sensing the beat of the heart, and a pulse generator for sending the demand pacer pulses to the heart when the heart would otherwise pause to contract.

The pacer catheter is used by making an incision in a vein or by percutaneously puncturing a vein leading to the heart. The end of the probe with the pacer electrode is inserted into the vein and threaded to the heart and into the right ventricle. When the depolarization of the heart muscle is sensed via the pacer electrode(s), the sensed signal is carried up the lead to the controller. If the lead fails to deliver the signal from the heart, the controller senses the missing signal and triggers the pulse generator to transmit the electrical demand pacer pulse to the heart muscle via the lead's electrode. Once emitted from the electrode, the pulse stimulates the right ventricle, causing depolarization of the heart. The pulse current's circuit is completed via the return electrode.

The approaches for applying the different low and high charging electrical pulses require two procedures, one for inserting the pacer catheter and one for attaching the defibrillator electrical patch, which expose the subject to high-risk conditions and a long recovery period. However, the high risk of this operation can be avoided by using a “lead” for defibrillation that can enter the heart via a vein to replace the patch.

Moreover, a single catheter can be inserted into the heart for applying both defibrillator or demand pacer pulse to the heart depending on the condition of the heart.

Generally, the catheter for applying the defibrillation and demand pacer pulses has a flexible probe that can be inserted into a vein and threaded through the right atrium and into the right ventricle of the heart. A return electrode and a demand pacer electrode are attached to the portion of the probe in the right ventricle. A defibrillator electrode is attached to the portion of the probe in the right atrium. Connected to the other end of the probe is a regulator having a controller for sensing and analyzing the electrical pulses of the heart. The regulator can further include a defibrillator capacitor and demand pacer capacitor for transmitting their respective pulses to the heart. The capacitor for defibrillation is charged via a DC-AC converter powered by a battery located in the regulator. The regulator is inserted into the body, such as in the subcutaneous tissue of the chest wall, so that the system is independently contained within the body.

As the heart produces its electrical signal, it is sensed and sent to the controller. The controller then uses this information to determine if the heart is acting properly. If not, the controller automatically informs either the demand pacer or the defibrillator to transmit its respective pulse to the respective electrode. The pulses then travel through the blood and into the surrounding heart tissue, thereby defibrillating or demand pacing the heart. Finally, the charge returns to the implanted controller via the return electrode.

Certain arrhythmias may also be corrected by low energy stimulation in the form of a critically timed series of pulses to the heart. U.S. Pat. No. 5,690,682 discusses a device for treating cardiac arrhythmia including an implantable programmable drug delivery system for injection of a pharmaceutical agent into the peritoneum. U.S. Pat. No. 5,527,344 discusses a pharmacologic atrial defibrillator and method for automatically delivering a defibrillating drug to a subject. Likewise, U.S. Pat. No. 5,220,917 discusses an implantable pharmacological defibrillator with automatic recognition of ventricular fibrillation.

However, the efficacy of these forms of therapy depends on many factors. critical problems can still occur after successful defibrillation. There may be a very slow heart rate (bradycardia), the beat may be erratic (arrhythmia) or there may be no heart rate (cardiac standstill). At times there may be an electrical impulse, yet no effective mechanical contraction (electromechanical dissociation—EMD); these conditions are potentially life threatening even after the arrest or fibrillation has been successfully alleviated.

Although implantable defibrillators have increased survival from sudden death, a significant number of patients with implantable cardiovertor defibrillators still die prematurely. One of the contributing factor is that the implanted defibrillators do not perform their assigned function properly. The exact etiology is unknown, but may be either asystole or pulseless electrical activity (PEA). Since the onset of fibrillation or cardiac arrest is swift and causes damage to the subject in a short period of time, a fast response is necessary to ensure the effectiveness of these therapies. Intervention by an emergency medical team (EMT) or other medical assistance is often required immediately to assure survival. Unfortunately, a subject who has experienced cardiac arrest or fibrillation is unconscious, incapable of obtaining an EMT. Even if bystanders contact an EMT, the wait may be too long to ensure effective medical intervention.

Despite the many advances in the development of new drugs and devices for treating subjects with cardiac arrest, these drugs and devices in the prior art have had little or no positive effect on the survival rate, which is still less than 10%. One reason for the low survival rate is that successful treatment depends upon determining the correct cause for abnormal cardiac contractions or the lack of contractions and promptly responding to the episode.

### SUMMARY OF THE INVENTION

Whenever a person with a defibrillator/tachycardia controller experiences an episode when intervention is mandated in the form of stimulation, shock with or without an injection of a biochemical facilitator into the heart, the subject is in an unstable state and at high risk of death. In such a situation the person may require prompt medical attention and further interventions, which may be delivered either information about the condition of the subject as practical for making life-saving decisions. Furthermore, information about the subject's whereabouts must be available.

Accordingly, it is an object of the present invention to provide an implantable medical device to treat a condition that requires defibrillation, wherein the medical device detects via its fibrillation that is life threatening. The sensors monitor the electrical activity of the heart and other physical or chemical parameters, such as arterial oxygen, carbon dioxide tension, motion, intracardiac pressure, among many other possible parameters. The medical device further controls arrhythmia, and restores functions of a fibrillating heart in a subject and transmits data on the patient's heart condition so as to allow a physician to take charge of the situation immediately and provide therapies via telemedicine in a timely fashion. The device also transmits data on patient's location to an emergency team so that the patient can be located rapidly and can get immediate on-site help.

The medical device for monitoring and controlling arrhythmia, and for restoring functions of a fibrillating heart in a subject by therapies directed by a physician via two-way communication between the device and the physician, is comprised of (a) an implant; (b) a wearable unit comprising a programmer, and a position-locating module; (c) a first interface -for communication between the implant and the programmer; and (d) a second interface for two-way communication between a physician in an emergency team and the programmer through the position-locating module, wherein data on the location of the subject and the subject's heart conditions are sent to the physician for evaluation, and instructions for therapy are sent from the physician to the programmer.

Additional objects and advantages of the invention will be set forth in the description that follows, and in part will be obvious from the description, or may be learned by the practice of the invention. The objects and advantages of the invention can be realized and obtained by means of the instruments and combinations particularly pointed out in the appended claims.

### BRIEF DESCRIPTION OF THE DRAWINGS

FIG. 1 schematically illustrates the basic structure of the medical device.

FIG. 2 schematically illustrates one embodiment of the medical device. (10) sensors; (20) detector; (30) communication switch; (31) the implant sends activation signal to wearable unit; (35) wearable unit activates transmission via GPS and transmits data from implant; (36) activation of GPS; (40) capacitor; (42) determining effectiveness; (43) antitachycardia device; (45) shock enablement; (48) successful result; (49) the implant continues to monitor and analyze its inputs but remains silent; (50) metering pump (55) determining whether intervention is mandated; (60) stimulation applied; (65) No further action is needed and a record is made in the implant; (66) starts stimulation therapy, (70) determining whether automatic injection is required; (75) injection; (80) determining whether an expert should be contacted.

FIG. 3 schematically illustrates one embodiment of the configuration of the medical device. (10) implantable defibrillator for detecting and transmitting signals and for applying stimulation and/or shock; (200) wearable unit for receiving GPS transmission, controlling the implantable defibrillator and assessing the heart condition by analyzing data received from the implantable defibrillator, (300) Emergency team's station; (400) Mobile Unit.

FIG. 4 schematically illustrates the configuration of the medical device in more detail. (1) Sensing stimulating leads; (2) transmission antenna within the implantable defibrillator; (3) receiving antenna over the implantable defibrillator; (4) axis of line-up; (5) antenna for GPS reception and two-way RF communication, including radio beacon; (6) wearable receiver-transmitter unit; and (7) harness or belt to hold wearable unit.

### DETAILED DESCRIPTION OF THE INVENTION

The invention provides a medical device for monitoring and controlling arrhythmias, and for restoring functions of a fibrillating or arrested heart in a subject, and obtaining EMT intervention if necessary regardless of the location of the subject. The medical device for monitoring and controlling arrhythmia, and for comprising (a) an implant; and (b) a wearable unit comprising a programmer, and a position-locating module; (c) a first interface for communication between the implant and the programmer; and (d) a second interface for two-way communication between a physician in an emergency team and the programmer through the position-locating module, wherein data on the location of the subject and the subject's heart conditions are sent to the physician for evaluations, and instructions for therapy are sent from the physician to the programmer.

“Cardiac arrest” refers to a state where the heart of the subject has stopped beating and fails to provide blood circulation. Causes of cardiac arrest include but are not limited to heart diseases, generalized disease, anesthesia and surgical procedures. Cardiac arrest can also be “sudden death” that is, not apparently related to any underlying disease or preexisting condition.

“Cardiac condition,” “heart condition” or “heart disease” includes thrombosis, arrhythmia, and bradycardia, bradyasystole, cardiac standstill, electromechanical dissociation (EMD) and PEA.

Implantable sensor for blood oxygen level is disclosed in U.S. Pat. No. 6,122,536 (“the '536 patent”). The content of the '536 patent and the references cited in the '536 patent are incorporated herein by reference.

In one embodiment of the present invention, the medical device transmits data on the location of the subject to the emergency team to locate and to provide prompt medical treatment to the subject, wherein the data is used as navigational aid.

In another embodiment of the present invention, the implant of the medical device comprises (a) a sensor module for monitoring the conditions of a heart; (b) a defibrillating module for restoring the electromechanical function of the heart by means of stimulation and/or shock; (c) an optional injection module for optionally delivering a biomedical agent to the heart simultaneously with the stimulation or shock of step (b); (d) a data processing module for determining the types of interventions; and (e) an energy source for the sensor module, the defibrillating module, the injection module and the data-processing module.

In another embodiment of the invention, the first interface of the medical receiving signals from the sensor and for sending instructions to the defibrillator for action.

In another embodiment of the invention, the second interface of the medical device communicates with the emergency team through the position-locating module and a GPS satellite.

In another embodiment of the invention, the second interface of the medical device communicates with the emergency team through a mobile phone location system.

In another embodiment of the invention, the position-locating module of the medical device comprises a GPS receiver.

In another embodiment of the invention, the data processing module of the medical device makes a preliminary determination on choosing one kind of intervention selected from the group consisting of (a) applying stimulation or shock to the heart to restore its functions, (b) activating the second interface for help by transmitting data on patient's location and patient's heart condition to an emergency team, or (c) staying in a monitoring mode.

In another embodiment of the invention, the sensor module of the medical device monitors the electrical, physical and/or chemical parameters.

In another embodiment of the invention, the sensor module of the medical device monitors arterial oxygen, carbon dioxide tension, heart motion and/or intracardiac pressure.

In another embodiment of the invention, the subject is a human.

In another embodiment of the invention, the biomedical agent used in the medical device comprises a thyroid, or an adrenal hormone, wherein the agent enhances the responsiveness of the fibrillating heart.

In yet another embodiment of the invention, the thyroid hormone is selected from the group comprising thyroxine, triidothyronine, and thyroxine analogs.

In a further embodiment of the invention, the adrenal hormone is epinephrine or adrenaline.

As used herein, “VIP” and derivatives thereof refers to any natural VIP that activity. “Substantially similar” means a peptide in which amino acids nonessential to the VIP activity of the peptide have been altered in an attempt to change or enhance that activity, but the peptide still retains a high level of amino acid sequence similarity to the natural VIP peptide.

As used herein, thyroid hormones include, but not limited to the L-forms of thyroxine (3-(4-(4-Hydroxy-3,5-diiodophenoxy)-3,5-diidophenyl)-L-alanine or T4; hereinafter thyroxine) and 3, 5, 3′ triiodothyronine (hereinafter triiodothyronine or T3). T3 is qualitatively similar to thyroxine in its biological effect but is more potent on a molar basis. Although some T3 is synthesized in the thyroid gland, the majority of naturally occurring T3 is synthesized by metabolism of thyroxine in peripheral tissues by the enzyme 5′ deiodinase. A series of thyroxine analogs and methods of synthesis are described in U.S. Pat. No. 3,109,024.

The present invention also provides a method for emergency treatment of a subject with spontaneous cardiac arrest to restore the subject's effective cardiac function, comprising activating the medical device of claim 1 and applying therapy under the direction and control of a physician.

GPS is currently available in a variety of forms. “The basic GPS is defined as the constellation of satellites, the navigation payloads which produce the GPS signals, ground stations, data links, and associated command and control facilities which are operated and maintained by the Department of Defense; the Standard Positioning Service (SPS) as the civil and commercial service provided by the basic GPS; and augmentations as those systems based on the GPS that provide real-time accuracy greater than the SPS.” (Cited from: Positioning System Policy, The White House, Office of Science and Technology Policy, National Security Council, Mar. 29, 1996) The Department of Transportation serves as the lead agency within the U.S. Government for all Federal civil GPS matters (ibid.). In cooperation with the Departments of Commerce, Defense and State, the Dept. of Transportation takes the lead in promoting commercial applications of GPS technologies and the acceptance of GPS and U.S. Government augmentations as standards in domestic and international transportation systems. In cooperation with other departments and

The Absolute Positioning mode is defined with respect to a well-defined coordinate system, commonly a geocentric system (i.e., a system whose point of origin coincides with the center of mass of the earth). In inexpensive systems this method results in considerable spatial uncertainties on the order of 10 meters or more. For greater accuracy the Differential GPS—DGPS was developed. This allows the position to be defined more accurately by determining the positioning error at a specific location and then incorporating a “differential correction factor” by real-time transmission of corrections into the position calculations of a mobile receiver operating in the same area and simultaneously tracking the same satellites. The differential correction cancels fluctuating errors in the GPS signal by using another GPS receiver set up on a position with a known location. As used herein, “GPS” encompasses all embodiments.

Each EMT's home base can serve as a reference point. The receiver at that station computes its location from the continuously received GPS satellite data and compares this position with its known position. This difference is the local momentary error in the received GPS signal. The differential value is then used for correcting in real-time the positions collected by other GPS receivers during the same time period, observing the same satellites, such as the GPS receiver on the EMT's mobile unit and for correcting the error in the position transmitted by the fibrillation victim's wearable unit.

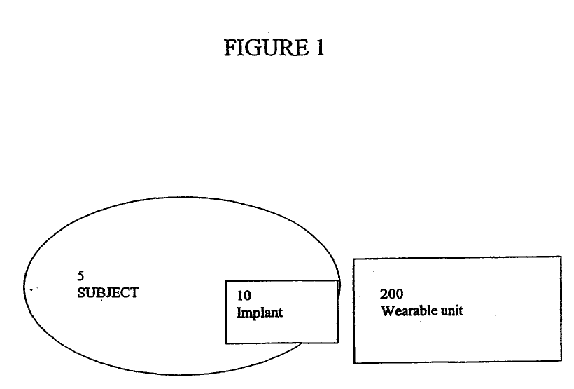

As shown in FIG. 1, communication between the implantable defibrillator and the wearable unit can either be one-way, only from the implantable defibrillator to the wearable unit, or bidirectional. However, all contemporary implantable defibrillators are equipped with electronics for two way communication at the site of the defibrillator, for receiving instructions from a “programmer” and sending information out about its operating parameters, its identity (manufacturer, model, serial number, etc.) and its past or current data flow (episodes observed, digitized electrograms stored from past episodes, and current electrograms and other inputs to the implantable defibrillator from its various sensors). Each implantable defibrillator thus has available at least two methods to send information to a nearby defibrillation shock through the subject's tissues. Thus, an implantable defibrillator may be modified to transmit information through a one-way (outbound) system the moment the implantable defibrillator recognizes an abnormal rhythm as defined by conditions pre-selected by the physician or as defined in the factory set default conditions.

The outbound transmission does not constitute any security issues. If the external, wearable system is able to receive the transmission then it will act accordingly, otherwise the implantable defibrillator simply “shouts into the wilderness” where its signal is not received. Such telemetry systems in implantable defibrillators can operate over a short range, a matter of a fraction of a meter, and use so little power for the transmission that they neither violate any regulations for radio frequency transmissions, nor impact significantly upon the life expectancy of the implantable defibrillator's energy source. The transmission of the shock itself does not constitute any additional energy requirement beyond that needed for the therapeutic goal. The wearable unit should have sufficient signal processing facilities to distinguish between a defibrillation shock and any other interference from its environment. This demands additional pattern recognition intelligence in the wearable unit.

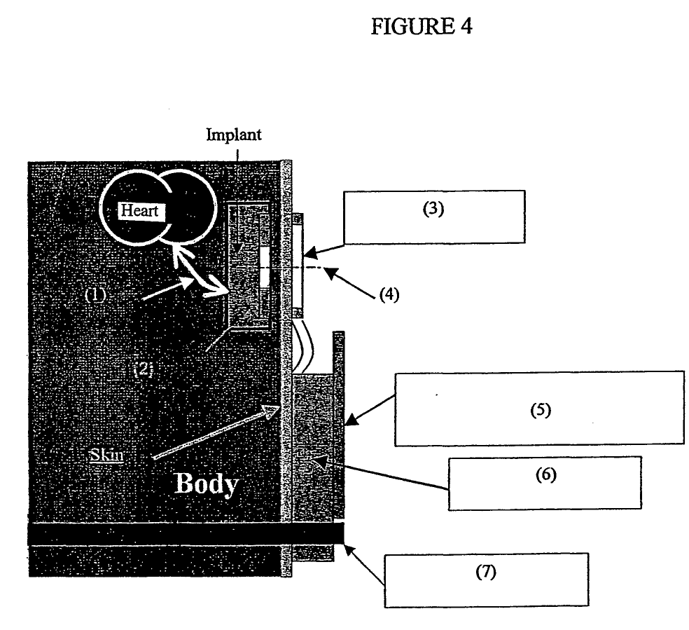

To assure that the wearable unit is never confused by receiving a signal from any other source than from the implantable defibrillator that it is assigned to monitor, two measures are preferably incorporated: the wearable unit has its receiving antenna for signals from the implantable defibrillator designed so that when it is donned, it will always be close enough to receive the signals from the implantable defibrillator. Such exclusive antenna pairs usually rely on the axial alignment of the implantable transmission coil antenna and the external coil antenna on the body surface as shown in FIG. 4.

In case communication is only outbound or one-way, from the implantable defibrillator to the wearable unit, then the sole purpose of the system is to alert the EMT about the crisis and assist the team to locate the victim. This may be accomplished through the GPS in combination with a radio beacon incorporated into

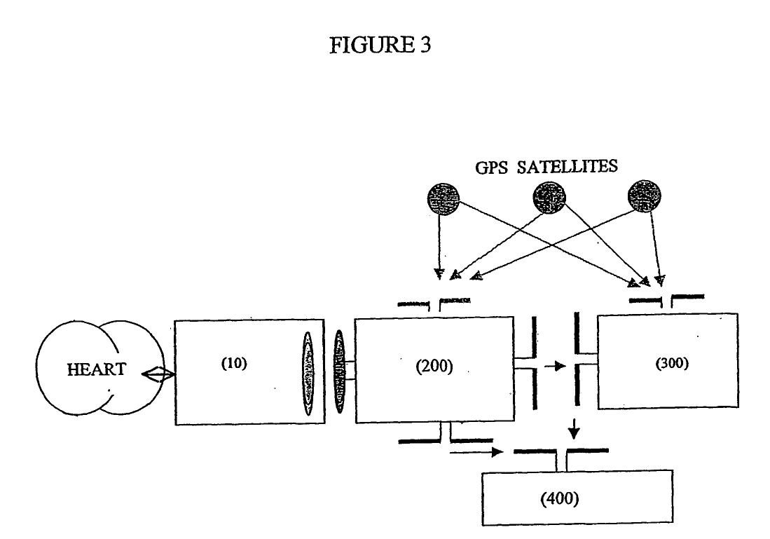

As indicated in FIG. 3, the antenna from the implanted defibrillator transmits the indication of a critical event to the antenna of the Wearable Unit. The security can be enhanced by the Wearable Unit having the ability to detect an identification code from the implantable defibrillator.

The Wearable Unit receives the GPS information and transmits its position, along with its own identification, to the EMT's Station (ETS). As this is a simple GPS system, the position may be inaccurate.

The ETS is always receiving its GPS information and its own absolute position is known, hence it can correct errors in the GPS data it receives. As it is close enough to the subject, it can also apply a correction to the data received from the Wearable Unit of the subject as it receives the transmission from the subject's Wearable Unit. Once the Mobile Unit (MU) is dispatched, the corrected position of the subject is transmitted from the ETS to the MU. The antenna represents the radio beacon on the Wearable Unit that transmits to the MU to aid the EMT in finding the subject. There are many ways to implement the radio beacon in conformance with FCC regulations and other restrictions.

If the system is enhanced by commands from an expert to the implantable defibrillator to control its actions, such as injection, energy level, etc. then an additional secure inward linkage must be established. That will be described in the next section in more detail.

The implant can also transmit processed data of its own calculations derived from its initial input. For instance, it may determine that fibrillation is absent but stimulation for the termination of the arrhythmia is called for. It can transmit the identification code for the arrhythmia encountered and recognized, and the code for the treatment in use.

WU represents a wearable unit consisting of (1a) a very specific electric field sensor to detect a message from the implant, or (2b) a sensor that detects the transient magnetic field of the current pulse issued into the patient's body to defibrillate or cardiovert the heart. The WU includes a mobile phone that is programmed to dial 911 upon detecting one of the two above-referenced inputs. The approximate coordinates for the victim. The WU also includes a radio beacon to assist the Mobile EMT team to locate the victim.

As a further variation, the WU communicates with the implant by having (1′a) a very specific field sensor to detect a message from the implant, or (2′b) a sensor that detects the transient magnetic field of the current pulse issued into the patient's body to defibrillate or cardiovert the heart. The WU includes a mobile phone that is programmed to dial 911 upon detecting one of the two above referenced inputs and then transmits permanent identification of the patient, the implant and other pertinent information, along with the telephone number or e-mail address of the EMTs' Center that is to be transferred there by the Mobile Service Provider along with the coordinates. The identification of the location of the caller is performed by the Mobile Service Providers, who then alerts the EMT center of the incident and provides approximate coordination for rescuing the victim, along with the added information. Moreover, the WU includes a programmer that is linked to the patient's mobile phone so as to establish two-way communication between the implant and the physician or EMT via a cellular phone link. Further, the WU includes a radio beacon to assist the Mobile EMT to reach the patient.

The implant can also be programmed noninvasively. As the needs in terms of the electrical parameters of an implant, such as rate of stimulation, stimulus strength, amplifier gain or “sensitivity” to detect biological signals, vary from patient to patient and even varies with time for an individual, the need for noninvasive adjustment of an implant was recognized and met by a variety of ways, including communication via coded magnetic pulses which could close a reed switch. Currently, the capabilities for altering an implant have increased enormously. The “programs” to be sent inward to an implant are chosen on the basis of data gathered by the implant itself about its own and the heart's performance. This information, including the electrogram continuously sensed by the implant may be telemetered outward by the implant to inform the physician as needed for fine-tuning the operation of the implant by programming. This is used in defibrillators and implantable pumps.

### EXAMPLE 1

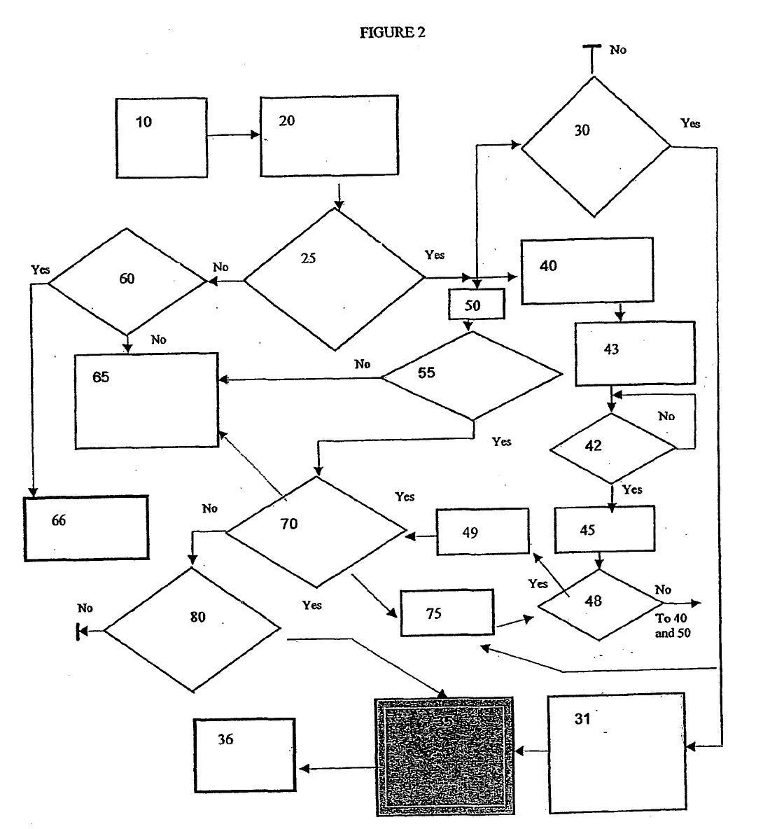

As shown in FIG. 2, the detector of the implant detects a chaotic episode from its sensors (10) that may be life threatening. The detector (20) through the input from its sensors (10) monitors the electrical activity of the heart and other physical or chemical parameters, such as arterial oxygen, carbon dioxide tension, motion, intracardiac pressure, among many other possible parameters. The decision is made in block 25 whether the event is fibrillation or not. If fibrillation is detected then the implant is either programmed to start communication block 30 of the detection of the event to the emergency team, or it merely continues to assess the incoming information until it recognizes the need for intervention by enabling the charging of the capacitor in block 40.

If communication is to be initiated at this moment, then the link needs be established between the implantable defibrillator 10 and a wearable transponder/transmitter/repeater or programmer 200 on the body of the subject 5. The wearable device 200 then attempts to establish a link with the emergency team automatically, as described below in some detail. Block 40 of the implant can also begin charging its delivery systems for intervention (charging a capacitor or filling a metering vessel block 55 with a chemical agent. If a chaotic condition is detected in Block 20 but it is classified as not fibrillation in block 25, then a further decision is made in block 60 whether to administer antiarrhthymic pacing. If that is appropriate, then the preselected stimulation protocol is implemented by block 65. If stimulation is not required, then the implant continues to monitor (block 40) and analyze its inputs but remains silent. Either the chaotic condition stops spontaneously—the crisis is over or action is postponed until the next message from block 20 appears. If communication had begun, it should be terminated according to a specific protocol (see below). The implant makes a record in its memory (65) and returns to monitoring. If fibrillation continues to be detected by block 25, the implant determines that intervention is necessary according to its operating rules and prepares for delivery. If the intervention is indicated by block 70 then it is carried out in block 75. If the implant is unable to determine whether to intervene or not shock. If a shock is indicated as the treatment according to the operating rules, then the capacitor's state is queried (42) until it is ready and then the shock is enabled in block 45. If the shock is successful (48) then the implantable defibrillator makes a record and returns to monitoring. If unsuccessful then the next intervention are enabled. If communication had started then it is now terminated (D.3). If instruction from the WU are present then the implantable defibrillator receives instructions for intervention and executes those or executes the default action after T seconds action (D3.1). If the default intervention is successful then return to monitoring; If the default intervention is unsuccessful as in (D-4), then the operating rules determine the next steps, including repeat shock intervention at the next higher intensity; (40-45) or deliver fluid into the heart and repeat shock intervention at the next higher level (40-45)(E). If the chaotic condition is not resolved at this point, the implantable defibrillator activates the wearable device to transmit the SOS signal to the emergency team. Meanwhile the implantable defibrillator continues the protocol as programmed; (F) the wearable device becomes a radio beacon to assist the emergency team in locating the subject.

### EXAMPLE 2

The implanted defibrillator first detected a fibrillation episode from its sensors that may be life threatening The sensors monitored the electrical activity of the heart and other physical or chemical parameters, such as arterial oxygen, carbon dioxide tension, motion, intracardiac pressure, among many other possible parameters. The implanted defibrillator was either programmed to start communication of the detection of the event to the EMT, or it merely continued to assess the incoming information until it recognized the need for intervention. If communication was initiated at this moment, then the link was established between the implant and a wearable transponder/transmitter/repeater or a programmer on the body of the subject. The wearable unit then attempted to establish a link with the EMT automatically. The implanted defibrillator began charging its delivery systems for intervention (charging a capacitor or filling a metering vessel with a pharmaceutical agent or both). only if initial attempts fail. The decision to shock with the maximal intensity (or maximal energy) and/or to inject a bolus of the pharmaceutical agent into heart from an implantable reservoir can be made before, during or after these initial events by a qualified physician or highly trained emergency medical technician. The link between that decision-maker and the implanted device, or the subject, if conscious, who had undergone such an episode, was established promptly and with high reliability. An immediate link between the decision maker and the device or the wearer of the device was also established at this time to achieve optimal results. The present GPS was used to rapidly establish the link.

### EXAMPLE 3

The transmission of data on both the patient's location and on the patient's heart conditions is accomplished by replacing the GPS of example 1 with a mobile telephone location system. A mobile telephone location system is disclosed in Sheffer et al., U.S. Pat. No. 5,844,522. The '522 patent uses a reverse voice channel signal, which is always on, as a tracking beacon for a field direction finder unit to track the x, y and z coordinate position quickly and accurately. The use of the devices illustrated in the above examples results in immediate response to cardiac arrest of a patient in two ways. At the onset of cardiac arrest, the claimed medical device transmits data on both the patient's heart condition and the patient's location to an emergency team or to a physician either directly or via a mobile-telephone system. An immediate response to the heart problem can be provided in the following two ways. (1) the transmitted data on the patient's heart condition allows a physician to take charge of the situation immediately and provide therapies via telemedicine in a timely fashion; and (2) using the transmitted data on patient's location as navigational aid, an emergency team can immediately locate the patient to provide on site help. Due to the immediate response, using these devices, the percent of survivors among patients experiencing cardiac arrest is expected to increase significantly, especially for those who live far away from medical centers or from physicians. It has been reported recently on New York Time that placing external defibrillators in sports arenas and on the planes of American Airlines had illustrates the importance of immediate attention by skilled personnel using defibrillation.

All references cited herein, both supra and infra, are hereby incorporated by reference. Although the foregoing invention has been described in some detail by way of illustration and example for the purposes of clarity and understanding, it will be apparent to those skilled in the art that certain changes and modifications can be practiced. Therefore, the description and examples should not be construed as limiting the scope of the invention, which is delineated by the appended claims.

1. A medical device for monitoring and controlling arrhythmias, and for restoring functions of a fibrillating heart in a subject by therapies directed by a physician via two-way communication between the device and the physician, comprising
(a) an implant;
(b) a wearable unit comprising a programmer, and a position-locating module;
(c) a first interface for communication between the implant and the programmer; and
(d) a second interface for two-way communication between a physician in an emergency team and the programmer through the position-locating module, wherein data on the location of the subject and the subject's heart conditions are sent to the physician for evaluation, and instructions for therapy are sent from the physician to the programmer.

2. The medical device of claim 1, wherein the data on the location of the subject is a navigational aid to the emergency team to locate and to provide prompt medical treatment to the subject.

3. The medical device of claim 1, wherein the implant comprises
(a) a sensor module for monitoring the conditions of a heart;
(b) a defibrillating module for restoring the electromechanical function of the heart by means of stimulation and/or shock;
(c) an optional injection module for optionally delivering a biomedical agent to the heart simultaneously with the stimulation or shock of step (b);
(d) a data processing module for determining the types of interventions;
and
(e) an energy source for the sensor module, the defibrallating module, the injection module and the data-processing module.

4. The medical device of claim 1, wherein the first interface is for two-way communications between the programmer and the implant for receiving signals

5. The medical device of claim 1, wherein the second interface communicates with the emergency team through the position-locating module and a GPS satellite.

6. The medical device of claim 1, wherein the second interface communicates with the emergency team through a mobile phone location system.

7. The medical device of claim 1, wherein the position-locating module comprises a GPS receiver.

8. The medical device of claim 2, wherein the data processing module decides the kinds of interventions selected from (a) applying stimulation or shock to the heart to restore its functions, (b) activating the second interface for help, or (c) staying in a monitoring mode.

9. The medical device of claim 2, wherein the sensor module monitors the electrical, physical and/or chemical parameters.

10. The medical device of claim 8, wherein the sensor module monitors arterial oxygen, carbon dioxide tension, heart motion and/or intracardiac pressure.

11. The medical device of claim 1, wherein the subject is a human.

12. The medical device of claim 2, wherein the biomedical agent comprises a thyroid, or an adrenal hormone, wherein the agent enhances the responsiveness of the fibrillating heart.

13. The medical device of claim 14, wherein the thyroid. hormone is selected from the group comprising thyroxine, triidothyronine, and thyroxine analogs.

14. The medical device of claim 14, wherein the adrenal hormone is epinephrine.

15. A method for emergency treatment of a subject with spontaneous cardiac arrest to restore the subject's effective cardiac function, comprising activating the medical device of claim 1 and applying therapy under the direction and control of a physician.
